(19) United States
(12) Patent Application Publication
(10) Pub. No.: US 2026/0167934 A1
(43) Pub. Date: Jun. 18, 2026

(54) BIOLOGICAL TISSUE MODEL AND MANUFACTURING METHOD THEREOF

(71) Applicants: TOPPAN Holdings Inc., Tokyo (JP); The University of Osaka, Suita-shi, Osaka (JP)

(72) Inventors: Shiro KITANO, Taito-ku, Tokyo (JP); Michiya MATSUSAKI, Suita-shi, Osaka (JP); Zhengtian XIE, Suita-shi, Osaka (JP)

(73) Assignees: TOPPAN Holdings Inc., Tokyo (JP); The University of Osaka, Suita-shi, Osaka (JP)

(21) Appl. No.: 19/124,315

(22) PCT Filed: Oct. 12, 2023
(86) PCT No.: PCT/JP2023/037069

§ 371 Date: Apr. 25, 2025

(30) Foreign Application Priority Data
Nov. 1, 2022 (JP) 2022-175612

(51) Int. Cl. C12N 5/077; B29C 64/118; B29L 31/40; B33Y 10/00; B33Y 80/00

(52) U.S. Cl. CPC C12N 5/0657; B29C 64/118; B33Y 10/00; B33Y 80/00; B29K 2089/00; B29L 2031/40; C12N 2513/00; C12N 2533/54; C12N 2537/00; C12N 2537/10

(57) ABSTRACT

The present disclosure relates to a biological tissue model including two or more tissue pieces including a bioink printed material, and having a structure in which the tissue pieces adhere to each other, wherein the bioink contains fragmented extracellular matrix components.

### TECHNICAL FIELD

The present invention relates to a biological tissue model and a manufacturing method thereof.

### BACKGROUND ART

Various studies have been made so far on a method for forming a biological tissue model in vitro (Patent Literatures 1 to 2, Non Patent Literatures 1 to 2, etc.). Non Patent Literatures 1 to 2 discloses a device and the like for forming a tissue body in vitro using three-dimensional bioprinting.

### CITATION LIST

### Patent Literature

Patent Literature 1: Japanese Unexamined Patent Publication No. 2013-525077

Patent Literature 2: Japanese Unexamined Patent Publication No. 2020-202856

### Non Patent Literature

Non Patent Literature 1: T. Dvir et al., Adv. Sci. 1900344(2019)

Non Patent Literature 2: A. W. Feinberg et al., Science vol 365, Issue 6452, 482-487(2019)

### SUMMARY OF INVENTION

### Technical Problem

In the conventional method disclosed in Non Patent Literature 1 to 2, the size of the biological tissue model that can be prepared is small, and it cannot be said that the biological tissue model sufficiently has a function close to that of a living body. Therefore, application to transplantation applications and the like has been difficult. A main cause of difficulty in application to transplantation applications and the like is a small operating space of a device itself such as a 3D printer for shaping a tissue. In order to simply increase the operating space, it is essential to secure a large space, but it takes enormous cost and time and is not realistic.

An object of the present invention is to provide a novel biological tissue model using a tissue piece that can be formed by printing a bioink.

### Solution to Problem

The present invention provides the following inventions.
[1] The biological tissue model including two or more tissue pieces including a bioink printed material, the biological tissue model having a structure in which the tissue pieces adhere to each other, wherein the bioink contains fragmented extracellular matrix components.
[2] The biological tissue model according to [1], wherein the fragmented extracellular matrix component comprises a fragmented collagen component.
[3] The biological tissue model according to [1] or [2], wherein the fragmented extracellular matrix component has an average diameter of 100 nm or less.
[4] The biological tissue model according to any one of [1] to [3], wherein a content of the fragmented extracellular matrix component is 5 mg/mL or more and 30 mg/mL or less based on the total amount of the bioink.
[5] The biological tissue model according to any one of [1] to [4], wherein the structure in which the tissue pieces adhere to each other is a structure in which the tissue pieces adhere to each other with fibrin.
[6] The biological tissue model according to any one of [1] to [4], wherein the structure in which the tissue pieces adhere to each other is a structure in which the tissue pieces adhere to each other by a crosslinking reaction between a crosslinkable compound and a metal ion.
[7] The biological tissue model according to any one of [1] to [4], wherein the structure in which the tissue pieces adhere to each other is a structure in which the tissue pieces adhere to each other by a polymerization reaction of a photopolymerizable compound.
[8] A method for manufacturing a biological tissue model, including a step of adhering tissue pieces including a bioink printed material to each other, wherein the bioink contains fragmented extracellular matrix components.

### Advantageous Effects of Invention

According to the present invention, it is possible to provide a novel biological tissue model using a tissue piece that can be formed by printing a bioink.

### BRIEF DESCRIPTION OF DRAWINGS

FIG. 1(A) is a photograph showing a biological tissue model prepared by adhering a bioink printed material on which human umbilical vein endothelial cells (HUVEC) or human dermal fibroblasts (NHDF) are arranged, FIG. 1(B) is a photograph showing an observation result of HUVEC on a biological tissue model, and FIG. 1(C) is a photograph showing an observation result of NHDF on a biological tissue model.

FIG. 2(A) is a photograph showing an observation result of cells on a biological tissue model, and FIG. 2(B) is an enlarged photograph in a frame shown in FIG. 2(A).

FIG. 3(A) and FIG. 3(B) are photographs showing a boundary region between a bioink printed material on which HUVEC is arranged and a bioink printed material on which NHDF is arranged in a biological tissue model.

FIG. 4(A) is a photograph showing a tissue piece including a bioink printed material for preparing a left chamber model, and FIG. 4(B) shows a left chamber prepared by adhering the tissue piece.

FIG. 5(A) is a photograph showing a tissue piece including a bioink printed material for preparing a right chamber model, and FIG. 5(B) shows a right chamber prepared by adhering the tissue piece.

FIG. 6(A) is a photograph showing tissue pieces including bioink printed material for preparing a left corona model, and FIG. 6(B) shows the left corona prepared by adhering these tissue pieces.

FIG. 7(A) is a photograph showing a tissue piece including a bioink printed material for preparing a right corona model, and FIG. 7(B) shows a right corona prepared by adhering the tissue piece.

FIG. 8 is a photograph showing a cardiac model prepared by adhering tissue pieces.

FIG. 9(A) and FIG. 9(B) are photographs showing a structure obtained by adhering tissue pieces to each other by metal ion crosslinking.

FIG. 10(A) and FIG. 10(B) are photographs showing a method of adhering tissue pieces to each other by a polymerization reaction of a photopolymerizable compound to prepare a structure and a structure prepared by the method.

### DESCRIPTION OF EMBODIMENTS

Hereinafter, embodiments for carrying out the present invention will be described in detail. However, the present invention is not limited to the following embodiments.

### Biological Tissue Model

The biological tissue model according to the present embodiment includes two or more tissue pieces including a bioink printed material, and has a structure in which the tissue pieces adhere to each other. The biological tissue model is a model that mimics the structure or function of an organ or a part of an organ. The biological tissue model is a biological tissue model that can be formed in vitro. Examples of the organ include a heart, a stomach, a small intestine, a large intestine, a vena cava, and an aorta. The biological tissue model may be a cardiac model (full-size cardiac model) or a model of a portion of the heart.

### <Tissue Piece>

The tissue piece comprises a bioink printed material. The tissue piece can be obtained by a method including printing the bioink using three-dimensional bioprinting or the like to form a printed material. The bioink printed material may be used as it is as a tissue piece, or may be subjected to crosslinking treatment or the like as necessary.

A bioink is an ink composition that can form a structure by bioprinting. Specifically, the bioink is an ink composition that contains a biocompatible material, is in a liquid state at the time of being discharged by the printer, and solidifies by stimulation, a lapse of time, or the like after being discharged from the printer.

The bioink contains a fragmented extracellular matrix component. The “fragmented extracellular matrix component” herein can be obtained by fragmenting the extracellular matrix component. The fragmented extracellular matrix components may be dispersed in the bioink.

The extracellular matrix component is an assembly of extracellular matrix molecules formed by a plurality of extracellular matrix molecules. The extracellular matrix molecule may be a substance present outside the cell in a multicellular organism. As the extracellular matrix molecule, any substance can be used as long as it does not adversely affect the growth of cells and the formation of cell assemblies. Examples of the extracellular matrix molecule include, but are not limited to, collagen, laminin, fibronectin, vitronectin, elastin, tenascin, entactin, fibrillin, and proteoglycan. As the extracellular matrix component, these extracellular matrix molecules may be used singly or in combination of two or more kinds thereof.

The extracellular matrix molecule may be a modification and variant of the extracellular matrix molecule described above, or may be a polypeptide such as a chemically synthesized peptide. The extracellular matrix molecule may have a repeat of a sequence represented by Gly-X-Y characteristic of collagen. Here, Gly represents a glycine residue, and X and Y each independently represent an arbitrary amino acid residue. The plurality of Gly-X-Ys may be the same or different. By having the repetition of the sequence represented by Gly-X-Y, the restriction on the arrangement of molecular chains is reduced. In the extracellular matrix molecule having repetition of the sequence represented by Gly-X-Y, the proportion of the sequence represented by Gly-X-Y may be 80% or more, and preferably 95% or more, of the entire amino acid sequence. The extracellular matrix molecule may also be a polypeptide having an RGD sequence. The RGD sequence refers to a sequence represented by Arg-Gly-Asp (arginine residue-glycine residue-aspartic acid residue). Examples of the extracellular matrix molecule containing a sequence represented by Gly-X-Y and an RGD sequence include collagen, fibronectin, vitronectin, laminin, and cadherin.

Examples of collagen include fibrous collagen and non-fibrous collagen. Fibrous collagen means collagen as a main component of collagen fibers, and specific examples thereof include type I collagen, type Il collagen, and type III collagen. Examples of the non-fibrous collagen include type IV collagen.

Proteoglycans include, but are not limited to, chondroitin sulfate proteoglycans, heparan sulfate proteoglycans, keratan sulfate proteoglycans, and dermatan sulfate proteoglycans.

The extracellular matrix component may contain at least one selected from the group consisting of collagen, laminin, and fibronectin, and preferably contains collagen. The collagen is preferably fibrous collagen, more preferably collagen type I. As the fibrous collagen, commercially available collagen may be used, and specific examples thereof include collagen type I derived from pig skin manufactured by NH Foods Ltd.

The extracellular matrix component may be an animal-derived extracellular matrix component. Examples of animal species from which the extracellular matrix component is derived include, but are not limited to, human, porcine, and bovine. As the extracellular matrix component, a component derived from one kind of animal may be used, or components derived from a plurality of kinds of animals may be used in combination.

As used herein, “fragmentation” means making an assembly of extracellular matrix molecules smaller in size. The fragmentation may be performed under the condition of breaking the bond in the extracellular matrix molecule, or may be performed under the condition of not breaking the bond in the extracellular matrix molecule. The fragmented extracellular matrix component may contain a fibrillated extracellular matrix component (fibrillated extracellular matrix component) which is a component obtained by fibrillating the extracellular matrix component by application of a physical force. Fibrillation is an aspect of fragmentation, and is performed, for example, under conditions that do not break a bond in the extracellular matrix molecule.

The method for fragmenting the extracellular matrix component is not particularly limited. As a method for fibrillating the extracellular matrix component, for example, the extracellular matrix component may be fibrillated by applying a physical force such as an ultrasonic homogenizer, a stirring homogenizer, or a high-pressure homogenizer. In the case of using a stirring homogenizer, the extracellular matrix component may be homogenized as it is, or may be homogenized in an aqueous medium such as physiological saline. In addition, it is also possible to obtain a millimeter-sized or nanometer-sized fibrillated extracellular matrix component by adjusting the time, the number of times, and the like of homogenization. The fibrillated extracellular matrix component can also be obtained by fibrillating through repeated freezing and thawing.

The fragmented extracellular matrix component may at least partially comprise a fibrillated extracellular matrix component. The fragmented extracellular matrix component may consist only of the fibrillated extracellular matrix component. That is, the fragmented extracellular matrix component may be a fibrillated extracellular matrix component. The fibrillated extracellular matrix component preferably contains a fibrillated collagen component. The fibrillated collagen component preferably maintains a triple helical structure derived from collagen. The fibrillated collagen component may be a component that completely or partially maintains the triple helical structure derived from collagen.

Examples of the shape of the fragmented extracellular matrix component include a fibrous shape. The fibrous shape means a shape composed of a filamentous fragmented extracellular matrix component or a shape composed of a filamentous fragmented extracellular matrix component crosslinked between molecules. At least some of the fragmented extracellular matrix components may be fibrous. Fibrous extracellular matrix components include thin filaments (fine fibers) formed by aggregation of a plurality of filamentous extracellular matrix molecules, filaments formed by further aggregation of fine fibers, and fibrillated filaments thereof. In the fibrous extracellular matrix component, the RGD sequence is preserved without being destroyed.

The average length of the fragmented extracellular matrix component may be 100 nm or more and 400 μm or less, and may be 100 nm or more and 200 μm or less. In one embodiment, the average length of the fragmented extracellular matrix component may be 5 μm or more and 400 μm or less, 10 μm or more and 400 μm or less, 22 μm or more and 400 μm or less, or 100 μm or more and 400 μm or less. In other embodiments, the average length of the fragmented extracellular matrix component may be 100 μm or less, 50 μm or less, 30 μm or less, 15 μm or less, 10 μm or less, 1 μm or less, or 100 nm or more. The average length of most of the fragmented extracellular matrix components among the entire fragmented extracellular matrix components may be within the above numerical range. Specifically, the average length of 95% of the fragmented extracellular matrix components of the entire fragmented extracellular matrix components may be within the above numerical range. The fragmented extracellular matrix component may be a fragmented collagen component having an average length within the above range, or may be a fibrillated collagen component having an average length within the above range.

The average diameter of the fragmented extracellular matrix component may be, for example, 20 nm or more and 30 μm or less, or 20 nm or more and 10 μm or less. The fragmented extracellular matrix component having an average diameter of nano-order (1000 nm or less) is also referred to as nanofiber (NF). The average diameter of the nanofibers may be, for example, 20 nm or more and 1000 nm or less, 20 nm or more and 500 nm or less, 20 nm or more and 200 nm or less, 20 nm or more and 150 nm or less, 40 nm or more and 130 nm or less, or 20 nm or more and 100 nm or less. The fragmented extracellular matrix component having an average diameter in the micro-order (more than 1000 nm) is also referred to as a microfiber (MF). The average diameter of the microfibers may be, for example, more than 1 μm and 30 μm or less, more than 1 μm and 30 μm or less, more than 1 μm and 10 μm or less, 1.5 μm or more and 8.5 μm or less, or 2 μm or more and 8.5 μm or less. The fragmented extracellular matrix component may be a fragmented collagen component having an average diameter within the above range, or may be a fibrillated collagen component having an average diameter within the above range. In the present embodiment, the shapes of the nanofibers and the microfibers may not be fibrous as long as they are the above-described fragmented extracellular matrix.

The bioink may contain one or both of nanofibers and microfibers. When the bioink contains both nanofibers and microfibers, the nanofibers may be used more in terms of weight than the microfibers. The mass of the nanofibers based on the total mass of the fragmented extracellular matrix component in the bioink may be, for example, 80 to 100 mass %, 90 to 100 mass %, or 95 to 100 mass %.

The average length and average diameter of the fragmented extracellular matrix components can be determined by measuring individual fragmented extracellular matrix components with a light microscope and performing image analysis. In the present specification, the “average length” means an average value of the lengths in the longitudinal direction of the measured sample, and the “average diameter” means an average value of the lengths in the direction orthogonal to the longitudinal direction of the measured sample.

The particle size of the fragmented extracellular matrix component may be less than 40 μm. The fragmented extracellular matrix component having a particle size of less than 40 μm is a fragmented extracellular matrix component that passes through a filter having a pore size of 40 μm. When the particle size of the fragmented extracellular matrix component is less than 40 μm, the components in the bioink are less likely to aggregate, and the discharge by the 3D printer becomes smoother.

At least some of the fragmented extracellular matrix components may be intermolecularly or intramolecularly crosslinked. The fragmented extracellular matrix component may be crosslinked within the molecule constituting the fragmented extracellular matrix component, or may be crosslinked between the molecules constituting the fragmented extracellular matrix component.

The fragmented extracellular matrix component at least partially crosslinked intermolecularly or intramolecularly can be manufactured, for example, by a method including the step of crosslinking the fragmented extracellular matrix component (crosslinking step). The fragmented extracellular matrix components can include, for example, fragmented and crosslinked extracellular matrix components. The fragmented and crosslinked extracellular matrix component can be manufactured, for example, by a method including a step of fragmenting the extracellular matrix component and a step of crosslinking the fragmented extracellular matrix component in this order, or a method including a step of crosslinking the extracellular matrix component and a step of fragmenting the crosslinked extracellular matrix component in this order.

Examples of the crosslinking method include physical crosslinking by application of heat, ultraviolet rays, radiation, or the like, and chemical crosslinking by a crosslinking agent, an enzymatic reaction, or the like, but the method is not particularly limited. The cross-linking may be cross-linking via covalent bonds.

When the fragmented extracellular matrix component comprises a fragmented collagen component, cross-links may be formed between collagen molecules (triple helical structure) or between collagen fibrils formed by collagen molecules.

The fragmented extracellular matrix components can be crosslinked, for example, by using a crosslinking agent. The crosslinking agent may be, for example, a crosslinking agent capable of crosslinking a carboxyl group and an amino group, or a crosslinking agent capable of crosslinking amino groups. The crosslinking agent may be, for example, at least one selected from the group consisting of an aldehyde-based crosslinking agent, a carbodiimide-based crosslinking agent, an epoxide-based crosslinking agent, and an imidazole-based crosslinking agent from the viewpoint of economic efficiency, safety, and operability. Examples of the crosslinking agent include water-soluble carbodiimides such as glutaraldehyde, 1-ethyl-3-(3-dimethylaminopropyl)carbodiimide hydrochloride, and 1-cyclohexyl-3-(2-morpholinyl-4 ethyl)carbodiimide sulfonate.

The content of the fragmented extracellular matrix component in the bioink can be appropriately set according to the shape, use, and the like of the biological tissue model to be formed. The content of the fragmented extracellular matrix component may be 1.0 mg/mL or more, 3.0 mg/mL or more, 5.0 mg/mL or more, 10.0 mg/mL or more, 12.0 mg/mL or more, 15,0 mg/mL or more, or 18.0 mg/ml or more, and may be 45.0 mg/ml or less, 40.0 mg/ml or less, 35.0 mg/ml or less, 30.0 mg/mL or less, or 25.0 mg/mL or less based on the total volume of the bioink since the strength of the tissue piece including the bioink printed material is further improved. The content of the fragmented extracellular matrix component is 5 mg/mL or more and 45 mg/mL or less, 5 mg/mL or more and 40 mg/mL or less, 5 mg/mL or more and 35 mg/mL or less, 5 mg/mL or more and 30 mg/mL or less, 5 mg/mL or more and 25 mg/mL or less, 10 mg/ml or more and 45 mg/mL or less, 10 mg/mL or more and 40 mg/mL or less, 10 mg/mL or more and 35 mg/mL or less, 10 mg/mL or more and 30 mg/mL or less, 10 mg/mL or more and 25 mg/mL or less, 15 mg/mL or more and 45 mg/ml or less, 15 mg/mL or more and 40 mg/mL or less, 15 mg/mL or more and 35 mg/mL or less, 15 mg/mL or more and 30 mg/mL or less, or 15 mg/mL or more and 25 mg/mL or less based on the total volume of the bioink since the strength of the tissue piece including the bioink printed material is further improved.

The bioink may further include a structure-forming material. The structure-forming material is a material that can form a structure by bioprinting. The structure-forming material does not contain a component corresponding to the fragmented extracellular matrix component. The structure-forming material may be dissolved or dispersed in the bioink. As the structure-forming material, a commercially available bioink material can be used. The type of the structure-forming material can be appropriately selected according to the shape, application, and the like of the structure. The structure-forming material may be, for example, an extracellular matrix component. The extracellular matrix component may be those exemplified above.

Specific examples of the structure-forming material include collagen, fibrin, chitosan, nanocellulose, polylactic acid (PLA), polycaprolactone (PCL), hydroxyapatite (HA), β-tricalcium phosphate (β-TCP), alginic acid, and gelatin methacryloyl.

The structure-forming material may be crosslinked. The structure-forming material may be crosslinked by, for example, a method such as physical crosslinking by application of heat, ultraviolet rays, radiation, or the like, or chemical crosslinking by a crosslinking agent, an enzymatic reaction, or the like. As the crosslinking agent, the above-described crosslinking agent can be used.

The content of the structure-forming material can be appropriately set according to the shape, application, and the like of the biological tissue model. The content of the structure-forming material may be, for example, 0.1 mass % or more, 0.2 mass % or more, or 0.5 mass % or more, and may be 10 mass % or less, 5 mass % or less, or 2 mass % or less based on the total mass of the bioink.

The bioink may comprise an aqueous medium. Examples of the aqueous medium include physiological saline such as phosphate buffered saline (PBS), sterile water, and pH buffers such as good buffer.

The bioink may or may not include cells. The bioink may further contain components (other components) other than the above-described components.

Various conditions such as pH and viscosity of the bioink can be appropriately set according to the composition of the bioink, the application of the biological tissue model, the printer to be used, and the like. The pH of the bioink may be, for example, 5.0 to 8.0, 6.0 to 8.0, or 6.5 to 7.5. The bioink containing the fragmented extracellular matrix component hardly causes gelation even under neutral or near-neutral conditions (For example, 6.0 to 8.0 or 6.5 to 7.5). Therefore, the bioink containing the fragmented extracellular matrix component is easy to form a printed material by the bioprinter even under neutral or near-neutral conditions.

The bioink can be obtained, for example, by a method including a step of mixing the fragmented extracellular matrix component and the structure-forming material in an aqueous medium.

The tissue pieces may or may not include cells. The tissue piece including the cells can be obtained, for example, by printing a bioink including the cells to form a printed material including the cells, or by seeding and culturing the cells on the bioink printed material.

The cell is not particularly limited, and may be, for example, a cell derived from a mammal such as a human, a monkey, a dog, a cat, a rabbit, a pig, a cow, a mouse, or a rat. The cell origin site is also not particularly limited, and may be a somatic cell derived from bone, muscle, visceral, nerve, brain, bone, skin, blood, or the like, or may be a germ cell. Further, the cell may be a stem cell, or may be a cultured cell such as a primary cultured cell, a subcultured cell, and a cell line cell.

The shape of the tissue piece and the bioink printed material is appropriately selected according to the shape of the biological tissue model. Examples of the shape of the bioink printed material include a sheet shape, a fiber shape, a spherical shape, a substantially spherical shape, an ellipsoid shape, a substantially ellipsoid shape, a hemispherical shape, a substantially hemispherical shape, a semicircular shape, a substantially semicircular shape, a rectangular parallelepiped shape, a substantially rectangular parallelepiped shape, a shape obtained by combining these shapes, and the like. The tissue piece and the bioink printed material may have a curved part, a through hole, or the like.

The thickness of the tissue piece is appropriately set according to the type of the biological tissue model and the like. The thickness of the tissue piece may be, for example, 0.1 cm or more, 0.3 cm or more, or 0.5 cm or more, and may be 4.0 cm or less, 3.0 cm or less, 2.5 cm or less, or 2.0 cm or less. The thickness of the tissue piece may be, for example, 0.5 cm or more and 2.0 cm or less. The thickness of the tissue piece may be the maximum distance in the thickness direction of the tissue piece.

The length or diameter of the long side when the biological tissue is viewed in plan view from the thickness direction may be, for example, 1 cm or more, 2 cm or more, or 3 cm or more, and may be 8 cm or less, 7 cm or less, 6 cm or less, or 5 cm or less. The length or diameter of the long side when it is viewed in plan view from the thickness direction may be, for example, 3 to 5 cm.

The number of tissue pieces and bioink printed materials in the biological tissue model is 2 or more, and may be 3 or more, 4 or more, 5 or more, 8 or more, 10 or more, 15 or more, 20 or more, 25 or more, 30 or more, 35 or more, 40 or more, 45 or more, 50 or more, or 55 or more. The number of tissue pieces and bioink printed materials in the biological tissue model may be, for example, 100 or less, 90 or less, 80 or less, 75 or less, 70 or less, or 65 or less.

The biological tissue model includes a structure in which tissue pieces adhere to each other. The structure in which the tissue pieces adhere to each other may be, for example, a structure in which the tissue pieces adhere to each other with an adhesive. As the adhesive, for example, an agent capable of being gelled after being applied to the adhesion site as a liquid can be used, and specific examples thereof include a mixture of fibrinogen and thrombin, a collagen solution, and the like.

Fibrinogen and thrombin are reacted with each other to form fibrin. Therefore, when tissue pieces are adhered to each other with a mixture of fibrinogen and thrombin, a part where the tissue pieces adhere to each other contains fibrin. The structure in which the tissue pieces adhere to each other is preferably a structure in which the tissue pieces adhere to each other with fibrin.

The content of fibrinogen in the mixture of fibrinogen and thrombin may be 30 to 70 mg/mL or 40 to 60 mg/mL, based on the total amount of the mixture. The content of thrombin in the mixture of fibrinogen and thrombin may be 10 to 30 units/mL or 15 to 25 units/mL, based on the total amount of the mixture.

The structure in which the tissue pieces are adhere to each other with an adhesive can be formed by a method in which an adhesive is applied to a part where the tissue pieces are adhered to each other to adhere the tissue pieces to each other.

The structure in which the tissue pieces adhere to each other may be a structure in which the tissue pieces adhere to each other by a crosslinking reaction between a crosslinkable compound and a metal ion. The crosslinkable compound is a compound having a crosslinkable group. Examples of the crosslinkable group include carboxy. The crosslinkable compound having a carboxy group is suitable as a compound that is crosslinked by a metal ion. As the metal ion, for example, an alkaline earth metal can be used, and specific examples thereof include a calcium ion, a magnesium ion, and a barium ion.

Examples of the crosslinkable compound include alginic acid and oxidized methacrylated alginic acid (OMA), Examples of the material that crosslinks the crosslinkable compound having a carboxy group include calcium chloride, magnesium chloride, and barium chloride.

The structure in which the tissue pieces are adhered to each other by a crosslinking reaction between the crosslinkable compound and a metal ion can be formed by a method including: containing the crosslinkable compound in the tissue pieces; and adhering the tissue pieces to each other by crosslinking the crosslinkable compound with a metal ion at a part where the tissue pieces are adhered to each other. The crosslinkable compound may be contained in the tissue piece by being contained in the bioink. The crosslinking by the metal ion may be performed by contacting a tissue piece containing a crosslinkable compound with a solution containing a metal ion. Specifically, by immersing tissue pieces containing a crosslinkable compound in a solution containing a metal ion, it is possible to form a structure in which the tissue pieces adhere to each other by a crosslinking reaction between the crosslinkable compound and the metal ion.

The structure in which the tissue pieces adhere to each other may be a structure in which the tissue pieces adhere to each other by a polymerization reaction of a photopolymerizable compound. The photopolymerizable compound is a compound having a polymerizable functional group. Examples of the polymerizable functional group include a methacryloyl group and an acryloyl group.

Examples of the photopolymerizable compound include oxidized methacrylated alginic acid (OMA).

The photopolymerizable compound may be polymerized by a photopolymerization initiator. Examples of the photopolymerization initiator include 2-hydroxy-2-methylpropiophenone.

The structure in which the tissue pieces are adhered to each other by the polymerization reaction of the photopolymerizable compound can be formed by a method in which a photopolymerizable compound and, if necessary, a photopolymerization initiator are contained in the tissue pieces, and at least a part where the tissue pieces are adhered to each other is irradiated with light to polymerize the photopolymerizable compound, thereby adhering the tissue pieces to each other. The photopolymerizable compound and the photopolymerization initiator may be contained in the tissue piece by being contained in the bioink.

The size of the biological tissue model can be appropriately selected according to the type, application, and the like of the target biological tissue model. The size of the biological tissue model may be an actual size of a biological organ or a part thereof, or a size obtained by appropriately changing the scale. The length of the biological tissue model in each of the longitudinal direction, the lateral direction, and the height direction (length×width×height) may be each 10 mm or more, 20 mm or more, 30 mm or more, 40 mm or more, 50 mm or more, 60 mm or more, 70 mm or more, 80 mm or more, 90 mm or more, or 100 mm or more, and may be 300 mm or less, 250 mm or less, 200 mm or less, 150 mm or less, or 120 mm or less. The biological tissue model may be, for example, a model (For example, a full size cardiac model) in which height×width×height is 50 to 120 mm×50 to 120 mm×50 to 120 mm.

Since the biological tissue model is formed by adhesion between a plurality of tissue pieces, a wide space is not necessarily required. Therefore, it is possible to easily produce a large biological tissue model without increasing the operating space of the device itself for forming the tissue piece.

In the tissue piece including the bioink printed material containing the fragmented extracellular matrix component, since the part where the tissue pieces adhere to each other is smooth, the biological tissue model can be more precisely prepared.

The biological tissue model can be suitably used as, for example, a scaffold material for cell culture or tissue formation, a substitute for experimental animals, or a transplantation material.

### Method for Manufacturing Biological Tissue Model

A method for manufacturing a biological tissue model includes a step of adhering tissue pieces including a bioink printed material (adhesion step). As a result, a biological tissue model including a structure in which tissue pieces adhere to each other is obtained. The method for adhering the tissue pieces may be as described above.

The method for manufacturing a biological tissue model may include a step of forming a tissue piece including a bioink printed material (printing step) before the adhesion step.

The printing step may be performed, for example, by a method including printing the bioink described above and solidifying the printed bioink. In this method, solidification may proceed while printing the bioink, and a precursor to a printed material having a desired shape may be formed by printing the bioink, and then the precursor to a printed material may be solidified to form the printed material.

The method of printing the bioink can be appropriately selected according to the use of the structure, the composition of the bioink, and the like. Specific examples of the method for printing the bioink include an inkjet method, a material extrusion method, a laser transfer method, and a stereolithography method.

The bioprinter may be a robotic bioprinter including standard components such as, but not limited to, a motor, a printhead, a printed circuit board, a printed structure, a cartridge, a syringe, a platform, a laser, and a control device. The bioprinter may be a 3D bioprinter such as INKREDIBLE (trademark), INKREDIBLE+ (trademark) or BIO X (trademark) manufactured by CELLINK AB.

Conditions for printing (For example, temperature, printing pressure, nozzle, and the like) can be appropriately set according to the shape and use of the printer and the structure. The temperature at the time of printing may be, for example, 4° C. or higher and 40° C. or lower. The printing pressure at the time of printing may be, for example, 1 kPa or more and 200 kPa or less.

The method of solidifying the printed bioink can be appropriately selected according to the composition of the bioink and the like. As a method of solidifying the printed bioink, a method of solidifying the bioink by a stimulus such as light or heat, a lapse of time, or contact with a liquid medium or the like can be used.

The printed bioink may be solidified in a support bath containing a liquid medium by being discharged from a printer or the like to the liquid medium. The liquid medium in the support bath can be appropriately set according to the composition of the bioink and the like. The pH of the liquid medium may be, for example, 5.0 to 8.0.

Examples of the liquid medium used for the support bath include a liquid medium in which particles are dispersed (hereinafter, referred to as a “particle dispersion medium”). When the liquid medium used for the support bath is a particle dispersion medium, the shape of the discharged bioink or the precursor of the structure formed by discharge is held by the particles in the liquid medium, so that the structure having high strength is more easily formed.

The particles in the particle dispersion medium may comprise biogum. Biogum means a polysaccharide produced by a biological body such as a microorganism or a plant. Examples of the biogum include microbial biogum and plant biogum. Examples of the microbial biogum include gellan gum, xanthan gum, diutan gum, welan gum, and pullulan gum. Examples of the plant biogum include acacia gum, tara gum, glucomannan, pectin, locust bean gum, guar gum, carrageenan, and tragacanth. The biogum may be gellan gum since it is particularly suitable for forming a high-strength structure.

The particle size of the particles in the particle dispersion medium may be, for example, 10 μm or more, 20 μm or more, 30 μm or more, or 40 μm or more, and may be 100 μm or less, 80 μm or less, or 60 μm or less. When the particle size is within the above range, formation of a structure having high strength is further facilitated. The particle size can be measured by an image using a confocal laser scanning microscope (For example, FV3000). Specifically, the particle size can be measured by acquiring an image of the particle dispersion medium using a confocal laser scanning microscope, performing image analysis by Image J, and manually calculating the particle size.

The particle size of the particles in the particle dispersion medium can be controlled by adding citric acid to the particle dispersion medium and adjusting the amount of citric acid added. When the particle dispersion medium contains citric acid, the content of citric acid may be more than 0 mol/L, 0.10 mol/L or more, 0.20 mol/L or more, or 0.25 mol/L or more, and may be 1.0 mol/L or less, 0.80 mol/L or less, 0.60 mol/L or less, or 0.40 mol/L or less, based on the total amount of the particle dispersion medium.

The particle dispersion medium containing biogum can be obtained, for example, by the following method. Biogum is dissolved in a liquid medium (For example, phosphate buffered saline) to obtain a biogum solution. The concentration of the biogum can be appropriately set according to the type of biogum and the like. When gellan gum is used as the biogum, the concentration of the biogum may be, for example, 0.3 to 0.7 mass % with respect to the total amount of the biogum solution. The biogum solution is gelatinized by performing a predetermined treatment required for gelatinization for each substance, such as allowing the biogum solution to stand for a predetermined time, thereby obtaining a biogum gel. The biogum gel is made into particles by homogenization to obtain a particle solution of biogum. A particle dispersion medium can be obtained by adding a citric acid buffer solution to a particle solution of biogum, further homogenizing the mixture, and centrifuging the mixture for degassing as necessary.

Another example of the liquid medium used for the support bath includes a liquid containing an organic solvent. When the bioink contains collagen as the structure-forming material, for example, a mixture of acetonitrile and water can also be used as the liquid medium used for the support bath.

The method for manufacturing a biological tissue model may further include a step (designing step) of designing a tissue piece to be formed by the printing step before the printing step. The designing step may include obtaining information of a biological tissue and dividing the biological tissue model into a plurality of tissue pieces. Acquisition of information on a biological tissue and division into a plurality of tissue pieces can be performed using a known database, software, and the like.

### EXAMPLES

Hereinafter, the present invention will be described more specifically based on Test Examples. However, the present invention is not limited to the following Test Examples.

### Preparation of Fragmented Collagen and Bioink

### <Material>·Collagen Type I Powder: (Nippi, Incorporated, PSC-1-200)

### <Procedure>

Type I collagen powder 50 mg was suspended in 5 mL of 10×PBS, homogenized at room temperature for 6 minutes using a homogenizer, and then centrifuged at 10,000 rpm for 5 minutes under the conditions of room temperature to obtain a fragmented collagen (CMF) solution containing CMF having an average diameter in the order of micrometers as a precipitate. The supernatant was removed from the solution, and 1×PBS was added thereto. The addition amount was 5 mL or 2.5 mL depending on the desired final concentration. Thereafter, the mixture was homogenized for 2 minutes and stored under an environment of 4° C. for 3 days to prepare a bioink containing fragmented collagen (CNF) having an average diameter on the order of nanometers. The final concentration of CNF in the bioink was adjusted as appropriate from the experiment.

The average diameter of the obtained fragmented collagen was 84.4±43.0 nm (number of samples: 25). The average diameter is calculated by measuring the fiber diameter distribution.

### Test Example 1: Preparation of Sheet-Shaped Biological Tissue Model

### <Preparation of Printed Material>

A sheet-shaped printed material (20 mm×10 mm×1 mm) was prepared by 3D printing the bioink having a CNF concentration of 10 mg/mL using a Bio-X printer according to an embedding printing method. As a support bath, granular gellan gum (GG) gel mixed with 0.3 M trisodium citrate (TSC) was used.

A pneumatic syringe equipped with a nozzle with a 25 G needle (inner diameter 250 μm) was filled with bioink. The printed materials designed by moving the nozzle according to a program while extruding bioink from the nozzle into the support bath were formed in the support bath.

The printing conditions were as follows.
Syringe pressure: 20 to 30 kPa
Nozzle moving speed: 25 mm/s
Filling density: 99%
Layer height: 0.1 mm

The printed materials were kept at room temperature for 1 hour such that the collagen component gelled in a support bath. The printed material was then crosslinked by immersing the printed material overnight in a 50% aqueous ethanol solution containing glutaraldehyde (GA) at a concentration of 0.25% as a crosslinking agent. The printed material was washed with an excess amount of MiliQ water to remove the crosslinking agent and then sterilized by immersing the resulting printed material in 70% aqueous ethanol for at least 30 minutes.

The obtained printed materials were repeatedly washed five times with PBS in a clean bench, and the solvent was replaced with PBS, before being used as a scaffold for culturing cells.

<Preparation of Printed Material on which Cells Are Arranged>

The sterilized printed material was cut into 5 mm×5 mm square shapes and placed at the bottom of a 24 well plate. Human umbilical vein endothelial cells (HUVEC) or human dermal fibroblasts (NHDF) were then seeded onto the printed materials at a cell density of about 1×10⁶/cm². Specifically, HUVEC or NHDF was dispersed in a medium, and the medium in which the cells were dispersed was added to the printed material. As a medium, KBM was used for HUVEC, and DMEM was used for NHDF. The printed material on which HUVEC or NHDF was disposed was cultured for 1 day under the conditions of 37° C. and 5% CO₂. As HUVEC, HUVEC (GFP-HUVEC) expressing GFP was used for observation.

### <Adhesion Between Tissue Pieces>

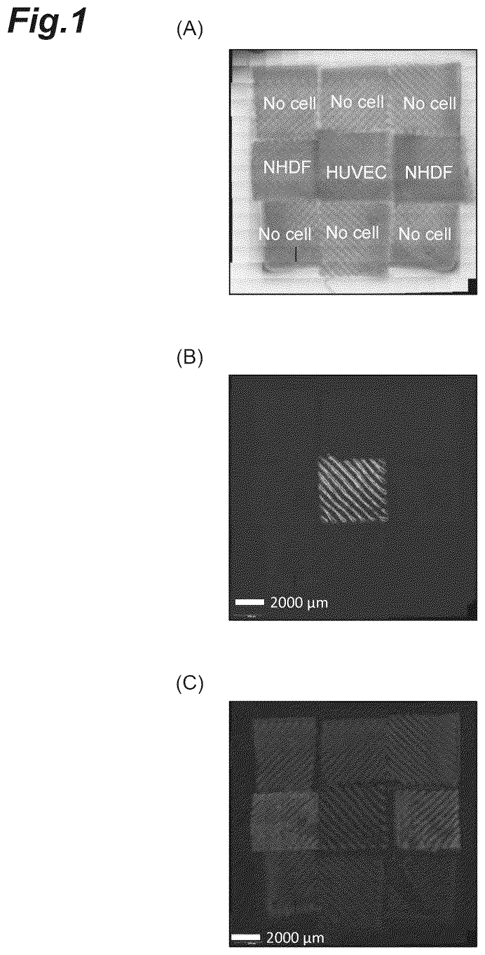

The printed material in which the cells were not arranged, the printed material in which the HUVEC was arranged, and the printed material in which the NHDF was arranged were used as tissue pieces, and the tissue pieces were assembled so as to have the pattern illustrated in FIG. 1(A), and adhesion between the tissue pieces was performed to obtain a biological tissue model of Example 1. The tissue pieces were adhered to each other by adding bio-glue (mixture of fibrinogen 50 mg/mL and thrombin 20 units/mL) to the part to be adhered and gelatinizing the bio-glue.

### <Cell Culture and Observation of Cell>

The biological tissue model of Example 1 prepared by adhering tissue pieces was placed in a 6-well plate. 5 mL of a mixed medium of KBM and DMEM (1:1) was added to the plate, and the biological tissue model of Example 1 was cultured under the conditions of 37° C. and 5% CO₂. After culturing for one week, the obtained culture was fixed with 4% paraformaldehyde (PFA), and immunostaining of CD31 and nuclei was performed, and cells on the biological tissue model of Example 1 were observed with a confocal laser scanning microscope (CLSM). NHDF was labeled with Cell Tracker (Deep Red).

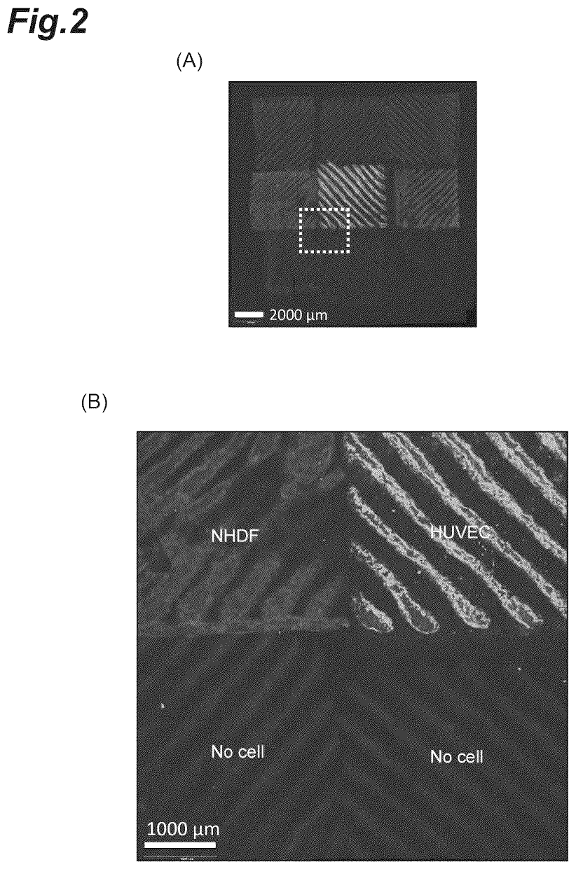

FIG. 1(B) illustrates HUVEC on a biological tissue model, FIG. 1(C) illustrates NHDF on a biological tissue model, and FIG. 2(A) and FIG. 2(B) illustrate observation results of a boundary region of a tissue piece. FIG. 1(B), FIG. 1(C) and FIG. 2(A), FIG. 2(B) show observation results on day 1 of culture.

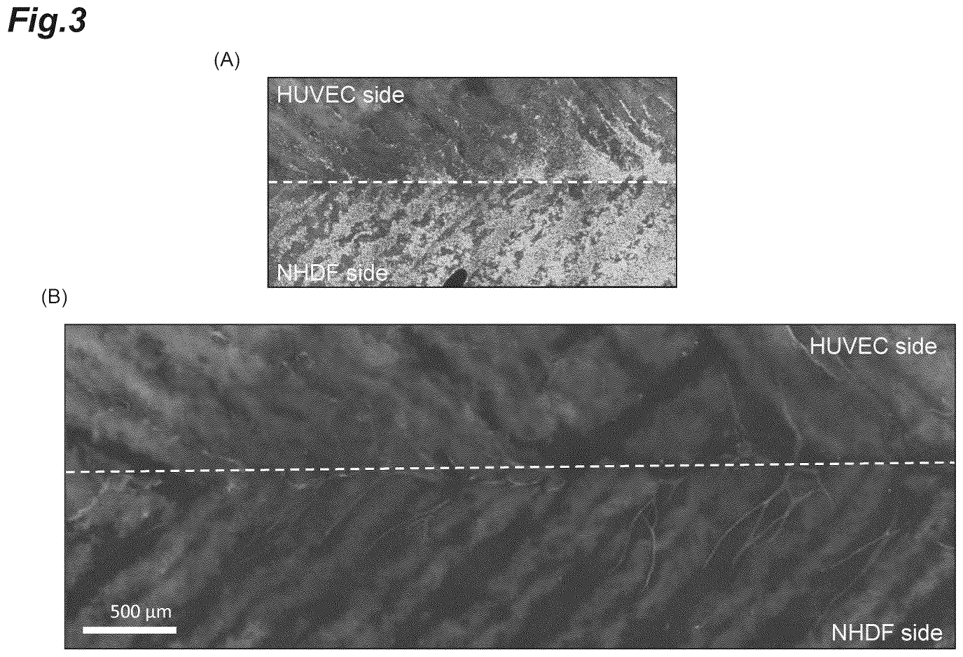

FIG. 3(A) and FIG. 3(B) are photographs showing observation results of a boundary region between a tissue piece containing HUVEC and a tissue piece containing NHDF. FIG. 3(A) and FIG. 3(B) show the observation results on day 7 of culture.

From FIG. 2(A) and FIG. 2(B), it can be seen that there is a clear boundary in the distribution of cells in the part where tissue pieces adhere to each other on day 1 of the culture period. FIG. 3(A) and FIG. 3(B) show that HUVECs form capillaries due to the influence of NHDF in the boundary region between tissue pieces on day 7 of the culture period.

### Test Example 2: Preparation of Cardiac Model

### <3D Model Design>

A full-size cardiac model (115 mm×118 mm×105 mm) was downloaded from BodyParts3D/Anatomography (URL: https://lifesciencedb.jp/bp3d/), which is a database in which the position and shape of each part of the human body are described as a three-dimensional model. The downloaded cardiac model was divided into tissue pieces less than 5 cm wide and modified to remove non-printable defects by the Autodesk Meshmixer (URL: https://www.meshmixer.com/). The obtained 3D model of each tissue piece was sliced with software Repetier-Host (URL: https://www.repetier.com/).

### <3D Printing of Each Tissue Piece of Full-Size Heart>

Bioink (CNF concentration in PBS=2% by mass) was loaded into a 25 G needled pneumatic syringe. Granular gellan gum (GG) gel mixed with 30% ethanol was used as a support bath. Each tissue piece designed (bioink printed material) was made by moving the nozzle according to the program while the ink was extruded into the support bath.

The printing conditions were as follows.
Syringe pressure 60 to 80 kPa
Nozzle moving speed 25 mm/s
Filling density: 60%
Layer height: 0.1 mm

Each tissue piece obtained by printing was held at room temperature for 3 hours so that the collagen component gelled. Each tissue piece was then crosslinked by immersing overnight in 50% aqueous ethanol with glutaraldehyde (GA) at a concentration of 0.25% as a crosslinking agent. The crosslinking agent was removed by washing with excess MiliQ water and each resulting tissue piece was stored in 70% ethanol.

<Adhesion between Tissue Pieces>

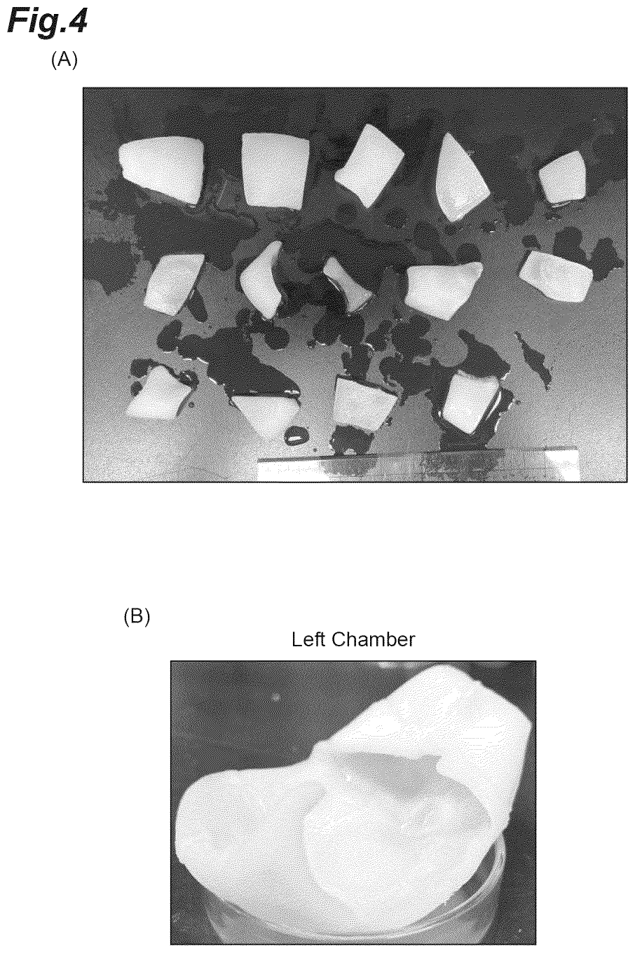

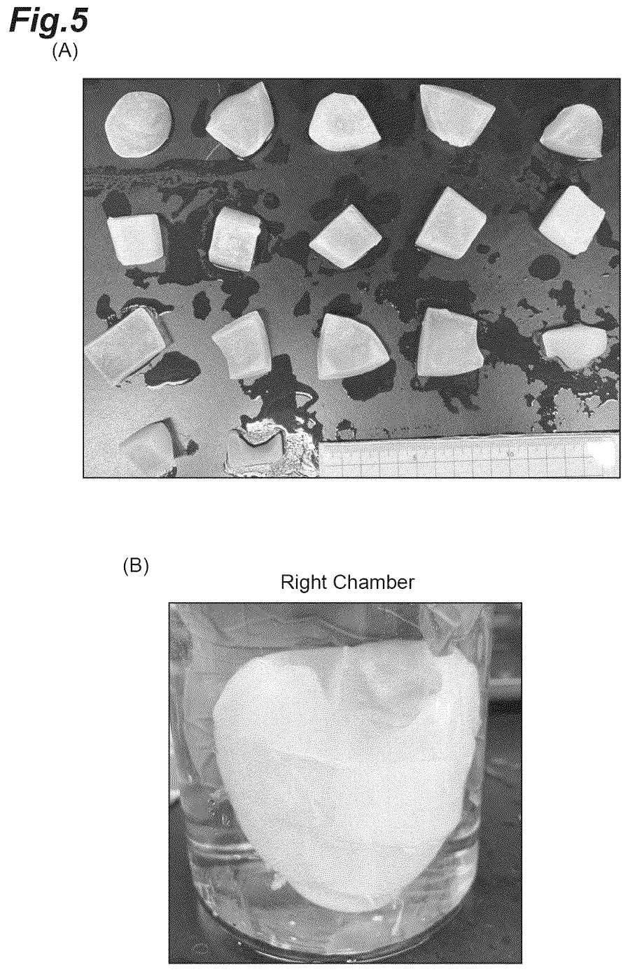

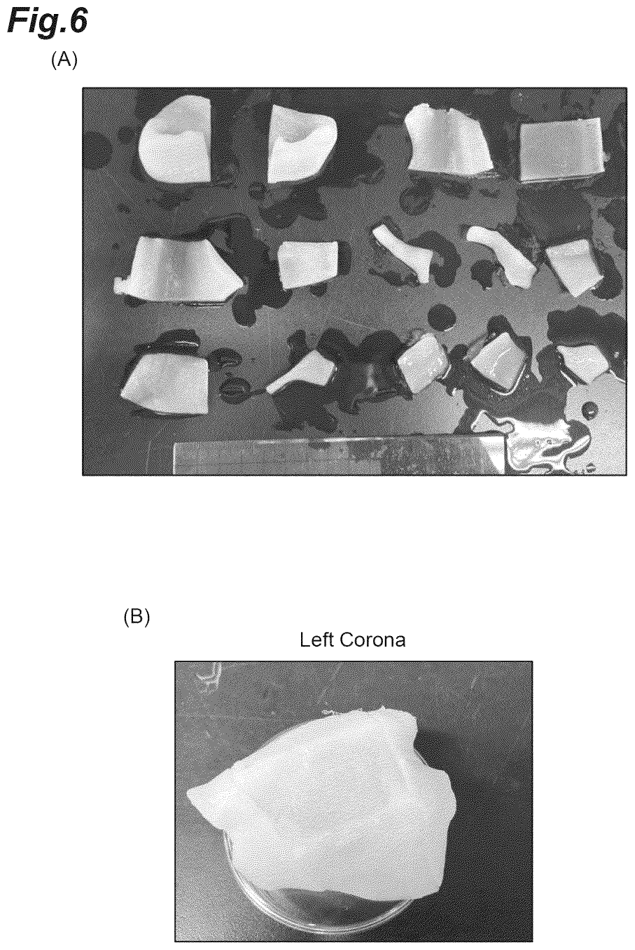

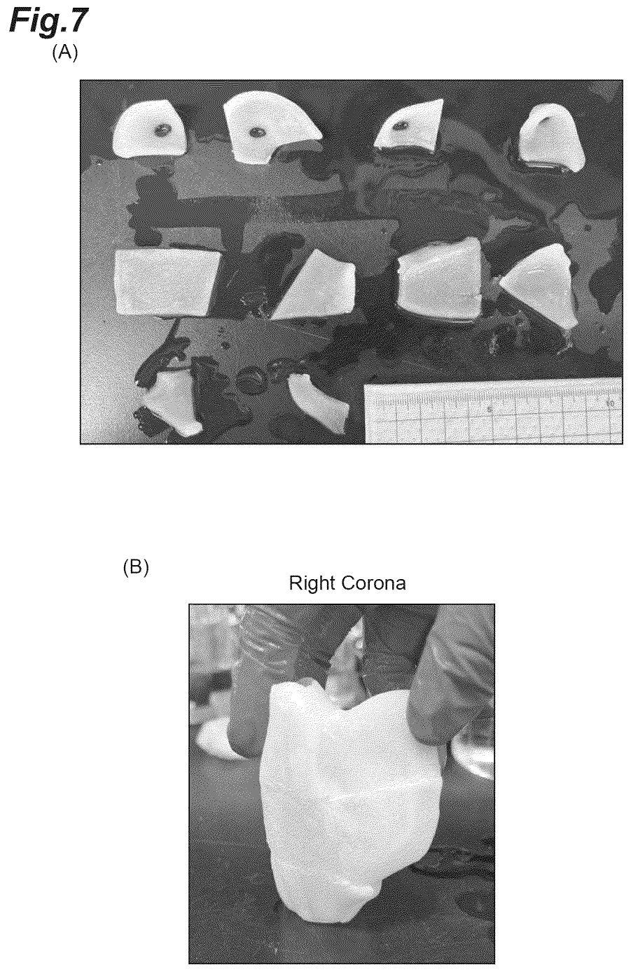

Each tissue piece was adhered by adding bio-glue (mixture of 50 mg/mL fibrinogen and 20 units/mL thrombin) to the part to be adhered. FIG. 4(A) shows tissue pieces for left chamber model preparation, and FIG. 4(B) shows the left chamber prepared by adhering these tissue pieces. FIG. 5(A) shows tissue pieces for right chamber model preparation, and FIG. 5(B) shows the right chamber prepared by adhering these tissue pieces. FIG. 6(A) shows tissue pieces for left corona model preparation, and FIG. 6(B) shows the left corona prepared by adhering these tissue pieces. FIG. 7(A) shows tissue pieces for right corona model preparation, and FIG. 7(B) shows the right corona prepared by adhering these tissue pieces.

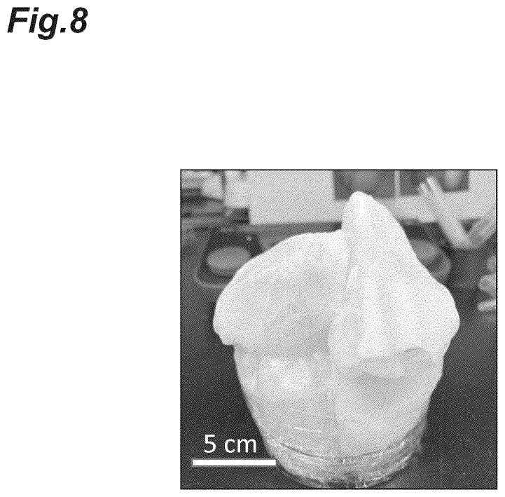

A left chamber, a right chamber, a left corona, a right corona, and other parts were assembled, and tissue pieces were adhered to each other to obtain a full-size cardiac model. FIG. 8 shows the obtained cardiac model. The resulting cardiac model was stored in 70% ethanol to inhibit bacterial growth. The total number of tissue pieces was 60 or more.

### Test Example 3: Preparation of Biological Tissue Model in Which Tissue Pieces Adhere to Each Other by Metal Ion Crosslinking

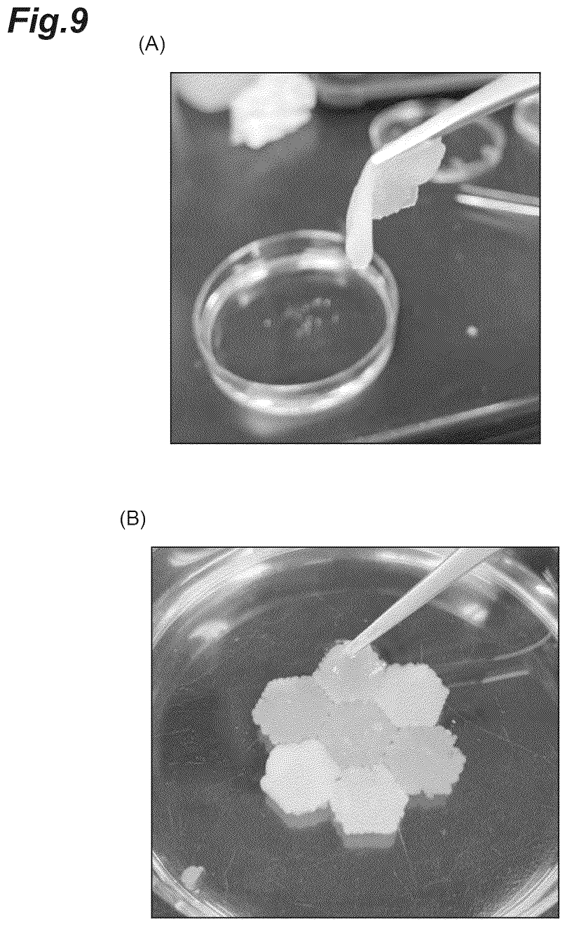

PBS containing 1 w/w % OMA and 1 wt % CNF was applied to the joint part as an adhesive in a state where seven sheet-shaped printed materials prepared and crosslinked in the same manner as in Test Example 1 except that the sheet shape was prepared in a substantially hexagonal shape with a side of 5 mm were combined as shown in FIG. 9(A) and FIG. 9(B). Thereafter, a 1 wt % CaCl₂ solution was further applied to the joint material and incubated at room temperature for 30 minutes. Thereafter, the tissue pieces adhered with tweezers were taken out, and it was confirmed that adhesion between the tissue pieces was well fixed as shown in FIG. 9(A) and FIG. 9(B).

### Test Example 4: Preparation of Biological Tissue Model in Which Tissue Pieces Adhere to Each Other by Polymerization Reaction of Photopolymerizable Compound

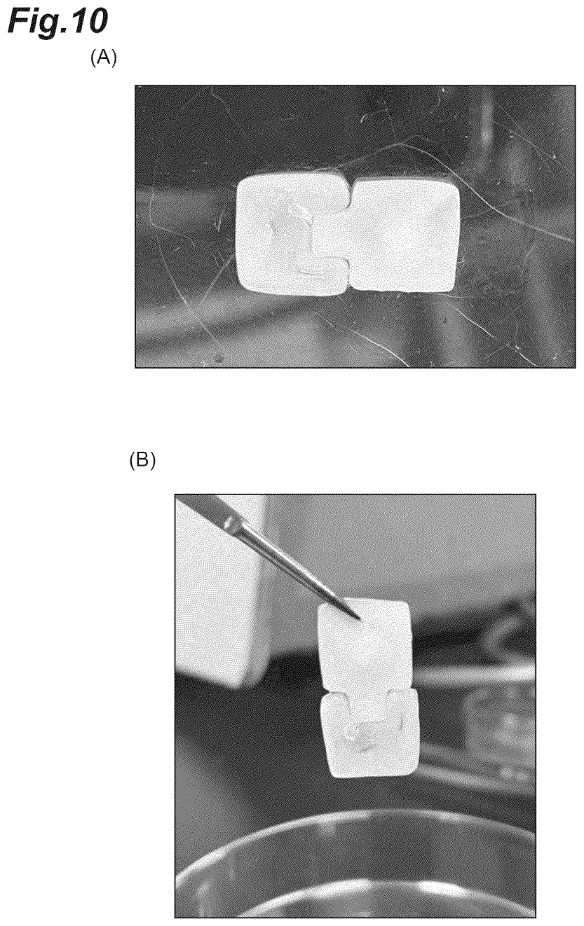

A sheet-shaped printed material prepared in the same manner as in Test Example 1 except that the sheet shape was formed into a substantially quadrangular shape of 10 mm on each side and a protrusion or a recess was provided in each of the shapes was fitted as shown in FIG. 10(A) and combined, and PBS containing 1 w/w % OMA, 1 wt % CNF, and 0.1 wt % 2-hydroxy-2-methylpropiophenone was applied as an adhesive to a joint part. Thereafter, the joint part was irradiated with ultraviolet rays having an intensity of 1 W/cm² for 10 minutes. Thereafter, the tissue pieces adhered with tweezers were taken out, and it was confirmed that adhesion between the tissue pieces was well fixed as shown in FIG. 10(B).

1. The biological tissue model comprising
two or more tissue pieces including a bioink printed material, the biological tissue model having
a structure in which the tissue pieces adhere to each other,
wherein the bioink contains fragmented extracellular matrix components.

2. The biological tissue model according to claim 1, wherein the fragmented extracellular matrix component comprises a fragmented collagen component.

3. The biological tissue model according to claim 1, wherein the fragmented extracellular matrix component has an average diameter of 100 nm or less.

4. The biological tissue model according to claim 1, wherein a content of the fragmented extracellular matrix component is 5 mg/mL or more and 30 mg/mL or less based on a total amount of the bioink.

5. The biological tissue model according to claim 1, wherein the structure in which the tissue pieces adhere to each other is a structure in which the tissue pieces adhere to each other with fibrin.

6. The biological tissue model according to claim 1, wherein the structure in which the tissue pieces adhere to each other is a structure in which the tissue pieces adhere to each other by a crosslinking reaction between a crosslinkable compound and a metal ion.

7. The biological tissue model according to claim 1, wherein the structure in which the tissue pieces adhere to each other is a structure in which the tissue pieces adhere to each other by a polymerization reaction of a photopolymerizable compound.

8. A method for manufacturing a biological tissue model, comprising
a step of adhering tissue pieces including a bioink printed material to each other,
wherein the bioink contains fragmented extracellular matrix components.

9. The method according to claim 8, wherein the fragmented extracellular matrix component comprises a fragmented collagen component.

10. The method according to claim 8, wherein the fragmented extracellular matrix component has an average diameter of 40 nm or more and 130 nm or less.

11. The method according to claim 8, wherein a content of the fragmented extracellular matrix component is 5 mg/mL or more and 30 mg/mL or less based on a total amount of the bioink.

12. The method according to claim 8, wherein the step of adhering the tissue pieces to each other comprises
containing a photopolymerizable compound in the tissue pieces, and
irradiating light onto at least the part where the tissue pieces are adhered to each other to polymerize the photopolymerizable compound and adhere the tissue pieces to each other.

13. The method according to claim 8, comprising containing a photopolymerization initiator in the tissue pieces in the step of containing the photopolymerizable compound.

14. The method according to claim 13, wherein
the photopolymerizable compound is oxidized methacrylated alginic acid, and
the photopolymerization initiator is 2-hydroxy-2-methylpropiophenone.

15. The method according to claim 8, wherein the step of adhering the tissue pieces to each other comprises
containing the crosslinkable compound in the tissue pieces; and
adhering the tissue pieces to each other by crosslinking the crosslinkable compound with a metal ion at the part where the tissue pieces are adhered to each other.

16. The method according to claim 15, wherein
the crosslinkable compound is at least one selected from the group consisting of alginic acid and oxidized methacrylated alginic acid; and
the metal ion is at least one selected from the group consisting of a calcium ion, a magnesium ion, and a barium ion.

17. The method according to claim 15, wherein
the crosslinkable compound is oxidized methacrylated alginic acid; and
the metal ion is a calcium ion.

18. The method according to claim 8, comprising the following steps before the step of adhering the tissue pieces to each other,
a step of printing the bioink, and
a step of solidifying the printed bioink,
wherein the step of solidifying the printed bioink comprises a step of solidifying the printed bioink in a support bath comprising a liquid medium by discharging the bioink to the liquid medium.

19. The method according to claim 18, wherein the support bath comprises granular gellan gum gel mixed with 0.3 M trisodium citrate.

20. The method according to claim 8, wherein the step of adhering the tissue pieces to each other is a step of adhering the tissue pieces to each other with fibrin.
